(19) United States
(12) Patent Application Publication
(10) Pub. No.: US 2022/0362437 A1
(43) Pub. Date: Nov. 17, 2022

(54) Hyaluronic Acid Compositions Containing Slowly Resorbable Polymers

(71) Applicant: Shanghai Qisheng Biological Preparation Co., Ltd., Shanghai (CN)

(72) Inventors: Dale P. DeVore, Chelmsford, MA (US); Jiaxun ZHU, Shanghai (CN); Changzheng WEI, Shanghai (CN); Lixia JIANG, Shanghai (CN); Peng DU, Shanghai (CN); Jianying WU, Shanghai (CN)

(21) Appl. No.: 17/744,319

(22) Filed: May 13, 2022

Related U.S. Application Data
(60) Provisional application No. 63/188,295, filed on May 13, 2021.

(51) Int. Cl. A61L 27/20; A61L 27/58; A61L 27/54; A61L 27/18

(52) U.S. Cl. CPC A61L 27/20; A61L 27/58; A61L 27/54; A61L 27/18; A61L 2300/402; A61L 2400/06

(57) ABSTRACT

Provided herein are hyaluronic acid compositions comprising hyaluronic acid or modified hyaluronic acid or crosslinked hyaluronic acid and slowly resorbable particles, spheres and granules. Also provided are methods for augmenting soft tissue utilizing the compositions.

### CROSS REFERENCE TO RELATED APPLICATION

This application claims the benefit of U.S. Provisional App. No. 63/188,295 filed May 13, 2021, which is incorporated by reference in its entirety for all purposes.

### FIELD OF INVENTION

The present invention describes compositions for augmenting soft tissue using injectable, hyaluronic acid or modified hyaluronic acid formulations containing bioresorbable biospheres or particles composed of polylactide (PLA) and polyglycolide (PLG) polymers and copolymers (PLGA) or poly ε-caprolactone (PCL) spheres or poly(p-dioxanone) (PDO) or polyglycolic acid (PGA) spheres or crosslinked hyaluronic acid microspheres or granules composed of other polymers such as L-Lactide/Trimethyl carbonate (Lactoprene®).

The supplemented hyaluronic acid compositions provide varying degrees of in vivo longevity dependent on the stability of the spherical particles, granules or nanoparticles.

### BACKGROUND

Use of bioabsorbable materials for facial soft tissue augmentation dates back to the early 1980s when bovine collagen was introduced to treat lines, wrinkles and volume defects. Since then, a variety of non-permanent, absorbable dermal fillers and facial implants have been approved and used worldwide (hyaluronic acid, collagen and porcine small intestinal submucosa). Semi-permanent and permanent dermal fillers have also been developed. Semi-permanent materials include hydroxylapatite (Radiesse) and poly L-lactic acid (Sculptra). Non-absorbable materials such as PMMA microspheres (Bellafill) and PTFE facial implant strands have also been used to correct facial defects.

The current markets for dermal fillers or products for soft tissue augmentation are dominated by products composed of hyaluronic acid. There are more than 110 hyaluronic acid fillers listed for the European market (Miinews). These HA fillers are crosslinked and differentiated primarily by concentration and HA particle size. However, none of the current HA products contain a supplement to enhance clinical durability.

Hyaluronic acid was discovered by Meyer and Palmer in 1934. Karl Meyer isolated the polysaccharide from the vitreous humor. Since it contained uronic acid, Meyer named the substance hyaluronic acid from hyalos (meaning glassy, vitreous) and uronic acid. At physiological pH, all carboxyl groups on the uronic acid residue are dissociated and the polysaccharide is named sodium hyaluronate when sodium is the counter ion. In 1986, Balazs suggested the name hyaluronan. This is currently the accepted terminology. HA is a linear polysaccharide (long-chain biological polymer) formed by repeating disaccharide units consisting of D-glucuronic acid and N-acetyl-D-glucosamine linked by β(1-3) and β(1-4) glycosidic linkages. HA is distinguished from other glycosaminoglycans, as it is free from covalent links to protein and sulphuric groups. It is, however, an integral component of complex proteoglycans. HA is an important component of the intercellular matrix, the material filling the space between the cells of such diverse tissues as skin, tendons, muscles and cartilage.

HA exhibits viscous flow, elastic and pseudoplastic properties. Those properties are unique to HA. Other glycosaminoglycans, GAGs, may form viscous solutions, but only at considerably greater concentrations than HA, and they never form a viscoelastic polymer network. HA has been demonstrated to be important in different activities such as tissue hydration, lubrication, solute transportation, cell migration, cell function, cell differentiation, and cell proliferation.

There are several methods to crosslink hyaluronic acid and other polysacccharides as discussed below. In addition, there have been literature publications and patents describing chemical crosslinking of collagen and hyaluronic acid including Rehakova et. al. (1996) using starch dialdehyde and Lin et. al. (2007) using 1-ethyl-3-(3-dimethylaminopropyl-carbodiimide (EDC) and U.S. Pat. No. 8,607,044 (Schroeder, et. al., 2014) using divinyl sulfone or 1,4-butanediol diglycidyl ether (BDDE).

There is a need in the art to develop hyaluronan materials for augmenting soft tissue.

### SUMMARY OF INVENTION

The present disclosure describes the application of crosslinked hyaluronic acid as a carrier for slowly resorbable particles, spheres and granules, such as PLA and PLGA spheres or poly ε-caprolactone (PCL) spheres or poly(p-dioxanone) (PDO) spheres and particles.

In some embodiments, the present disclosure relates to an injectable, chemically crosslinked hyaluronic acid solution containing resorbable poly-L-lactide (PLA) or poly-glycolide (PLG) lactide or L-lactide/glycolide copolymers-PLGA (20-50 μm diameter) or poly-ε-caprolactone (PCL) spheres or poly(p-dioxanone) (PDO) spheres for soft tissue augmentation and tissue regeneration.

In some embodiments, the chemically crosslinked hyaluronic acid may be purchased from a contract manufacturing organization or may be provided by a company producing and commercializing crosslinked hyaluronic acid for soft tissue augmentation. In addition, the crosslinked hyaluronic acid may be produced internally using existing and published procedures. For example, 1,4-butanediol diglycidyl ether (BDDE) may be utilized to produce crosslinked hyaluronic acid.

In some embodiments, poly-L-lactic acid crystals or microspheres may be procured from various manufacturers including Phosphorex, Carbion, Polysciences, or Akina. Poly-L-lactic acid crystals are milled and screened to provide microspheres or particles of 20-50 μm in diameter. Particles are sterilized using gamma irradiation, ethylene oxide, or other appropriate sterilization methods. Crosslinked hyaluronic acid is sterilized by autoclaving. Final product is prepared by aseptically combining the sterilized poly-L-lactic acid spheres or crystals with sterile crosslinked hyaluronic acid, followed by mixing to provide a homogeneous mixture and filling in containers (such as final product syringes).

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1: H&E stain of Control injected tissue in rabbit (40×).

FIG. 2: H&E stain of HA+PLA injected tissue in rabbit (40×).

FIG. 3: Trichrome Blue stain of Control injected tissue in rabbit (40×, arrow showed implantation and collagen fibrils are stained dark).

FIG. 4: Trichrome Blue stain of HA+PLA injected tissue in rabbit (40×, arrow showed implantation and collagen fibrils are stained dark).

FIG. 5: Geison's stain of Control injected tissue in rabbit (40×, showed implantation and elastic fibrils are stained dark).

FIG. 6: Geison's stain of HA+PLA injected tissue in rabbit (40×, showed implantation and elastic fibrils are stained dark).

### DETAILED DESCRIPTION OF THE INVENTION

All patents, patent applications, and literature references are hereby incorporated by reference in their entirety. The present invention provides a biologically compatible crosslinked hyaluronic acid composition supplemented with slowly degradable microspheres or crystals, such as those composed of poly-L-lactic acid, polyethylene glycol or copolymers of lactides and glycolides.

Unless otherwise specified, the term “hyaluronic acid” or its abbreviation “HA” will be used in this application in a broad sense to designate hyaluronan, or modified hyaluronic acid or crosslinked hyaluronic acid, or crosslinked hyaluronic acid microspheres and metallic salt thereof, such as sodium salt thereof.

As employed herein, the term “biologically compatible” refers to hyaluronic acid crosslinked or hyaluronic acid compositions formulated in accordance with the present invention which is stable when incorporated or implanted into or placed adjacent to the biological tissue of a subject and more particularly, does not deteriorate appreciably over time or induce an immune response or deleterious tissue reaction after such incorporation or implantation or placement.

As defined herein, the term “injectable hyaluronic composition” refers to an injectable, chemically modified or crosslinked compatible hyaluronic acid composition and such compositions supplemented with slowly resorbable microspheres which when injected into tissue, augments deficient tissue, such as skin lines and folds.

Several patents and patent applications are referenced (see Patent references) that describe the application of hyaluronic acid compositions for soft tissue applications. Several additional patents and patent applications describe the application of poly-L-lactides, polyethylene glycols and combinations thereof for potential application in drug delivery, application for tissue repair when combined with growth factors, or used alone (without addition to hyaluronic acid) for soft tissue augmentation. Still other patents and patent applications mention potential applications of bio-resorbable polymeric particles containing polymers of lactic acid. However, none of those patents or patent applications suggests to practicing a composition of crosslinked hyaluronic acid containing slowly resorbable particles, spheres and granules, such as poly-L-lactic acid microspheres or crystals for soft tissue augmentation. The only current soft tissue filler product containing microspheres or crystals of poly-L-lactic acid is Sculptra (FDA P020012 SSER) which is composed of carboxymethylcellulose, mannitol and PLLA in powder form.

The chemically crosslinked hyaluronic acid solution with slowly resorbable particles, spheres and granules can be injected into superficial dermis, mid-dermis, or deep dermis to correct contour defects in facial skin or can be injected into the loose connective tissue surrounding lip muscle or into the body of the lip to enhance lip appearance. The hyaluronic acid/slowly resorbable material compositions are injectable through a 25-30 gauge needle. The material basically remains colorless and provides a long-lasting clinical effect. The compositions can be prepackaged in ready-to-use syringes containing materials exhibiting several different degrees of durability.

To prepare hyaluronic acid compositions with extended durability, the crosslinked hyaluronic acid solutions are supplemented with 10-30% poly-L-lactide spheres (25-50 μm diameter), or particles of lactide/lactide or lactide/glycolide having similar dimensions. Polylactide compositions or polylactide/glycolide compositions (PLGA) provide soft dermal filler with extended durability compared to non-supplemented hyaluronic acid compositions.

The present disclosure is also directed to a method for augmenting soft tissue or regenerating tissue. The method comprises injecting the composition or the present disclosure into a soft tissue deficiency. The composition fills the soft tissue deficiency. As used herein, fill does not require that the deficiency be completely filled. However, completely filling the deficiency is also contemplated.

The method of soft tissue augmentation can be used with a variety of soft tissue deficiencies. Soft tissue defects that can be treated with the method or product of the invention include wrinkles, dermal folds, dermal laxity, skin contour defects, dermal fine lines, dermal furrows and dermal unevenness. The composition can be injected into a variety of dermal areas. The method is particularly well suited to be injected into a soft tissue deficiency of lips or facial skin. The amount of the composition to be injected can be determined by one skilled in the art based on the soft tissue deficiency being treated. In many applications, volumes of 0.1-0.5 mL may be used.

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described.

As used herein, the term “a” or “an” is intended to mean “one or more” (i.e., at least one) of the grammatical object of the article. Singular expressions, unless defined otherwise in contexts, include plural expressions. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The use of “or” means “and/or” unless stated otherwise.

As used herein, unless otherwise noted, the term “comprise”, “include” and “including” will be understood to imply the inclusion of a stated step or element or group of steps or elements but not the exclusion of any other step or element or group of steps or elements.

The phrase “consisting of” is meant to include, and is limited to, whatever follows the phrase “consisting of.” Thus, the phrase “consisting of” indicates that the listed elements are required or mandatory and that other elements may be present.

The following enumerated embodiments are representative of the invention:
1. A composition for use in soft tissue augmentation comprising
   (i) an HA carrier, wherein the carrier is selected from hyaluronic acid, modified hyaluronic acid, or crosslinked hyaluronic acid, or salts thereof (such as sodium salt); and
   (ii) slowly resorbable particles, spheres and granules.
2. The compositions of item 1, wherein the HA carrier has an molecular weight of from 1,000 to 10,000,000 Dalton, and/or the HA carrier is derived from animal tissue or microorganism fermentation.
3. The composition of item 1, wherein the modified hyaluronic acid or crosslinked hyaluronic acid are modified or crosslinked by divinylsulfone, glutaraldehyde, 1,4-butanediol diglycidyl ether, p-phenylene biscarbodiimide or 1,2,7,8-diepoxyoctane, or oligomers rich in amino groups (such as poly-lysine or poly-arginine or γ-polyglutamic acid); and/or
   the concentration of modified hyaluronic acid or crosslinked hyaluronic acid is from 12 mg/mL to 30 mg/mL, preferably from 16 mg/mL to 24 mg/mL; and/or
   the modified hyaluronic acid or crosslinked hyaluronic acid is cohesive.
4. The composition of item 1, wherein the crosslinked hyaluronic acid microspheres are coated with bio-degradable polymers, such as poly-L-lactide (PLA), polyethylene glycol (PEG), or PLGA, or poly(p-dioxanone) (PDO); and/or
   wherein the crosslinked hyaluronic acid microspheres are produced by emulsified crosslinking reaction, double emulsion evaporation method, microfluidic crosslinking reaction, or stamp formation; and/or
   crosslinker of the crosslinked hyaluronic acid microspheres is selected from the group consisting of divinylsulfone, glutaraldehyde, 1,4-butanediol diglycidyl ether or p-phenylene biscarbodiimide or 1,2,7,8-diepoxyoctane or oligomers rich in amino groups (such as poly-lysine or poly-arginine or γ-polyglutamic acid; and/or
   wherein the crosslinked hyaluronic acid microsphere has a particle size of particle size: 5˜150 μm, preferably 20˜50 μm.
5. The composition of item 1, wherein the slowly resorbable particles, spheres and granule spheres are selected from particles, spheres or granules of poly-L-lactide (PLA), PEG-PLA copolymer or poly-L-lactide-hydroxyapatite, polyglycolic acid (PGA), poly-L-lactide-hydroxyapatite, crosslinked hyaluronic acid microspheres.
6. The composition of item 1 wherein the slowly resorbable particles, spheres or granules are particles, spheres or granules of polylactide and polyglycolide polymers and copolymers (PLGA); and/or particles, spheres or granules of poly ε-caprolactone (PCL) or PCL-PLA copolymer or poly ε-caprolactone-hydroxyapatite microspheres; and/or poly(p-dioxanone) (PDO) or poly(p-dioxanone)-hydroxyapatite.
7. The composition of item 1, wherein the slowly resorbable particles, spheres or granules are produced through spray-precipitation technique, emulsion, double emulsion evaporation method, microfluidic reaction, Solid-Gel process, melt extrusion technique or stamp formation; and/or are sterilized through heat moist sterilization, gamma irradiation or ethylene oxide sterilization; and/or
   wherein the slowly resorbable particles, spheres or granules have a diameter of from 5 to 150 μm, preferably 20 to 50 μm.
8. The composition of item 1, further comprising additives, for example those selected from the group consisting of:
   local anesthesia drugs such as lidocaine, procaine, etc, preferably in a concentration of from 0.1% to 0.5% by weight; and/or
   polyols stabilizers, such as glycerin, mannitol, butanediol, sorbitol, preferably in a concentration of from 0.1 to 5% by weight; and/or
   a stabilizer with chelating ability, such as EDTA, EGTA, citric acid, sodium citrate, preferably in a concentration of from 0.1 to 5% by weight; and/or
   a sulfur stabilizer or dissolution promotor, such as Chondroitin Sulfate Sodium (CS), Gluscosamine Sulphate (GS) or Methyl sulfonyl methane (MSM), preferably in a concentration of from 0.1% to 5% by weight; and/or
   wherein soluble small molecules are added through dialysis process.
9. The composition of item 1, wherein the amount of HA carrier in the composition is from 0.1% to 55% by volume; and/or
   the amount of the slowly resorbable particles, spheres or granules in the composition is from 0.1% to 50% by volume, preferably from 1% to 20% by volume; and/or
   the additive in the composition is from 0.1% to 10% by weight, preferably from 0.1% to 5% by volume.
10. The composition of item 1, wherein the composition comprises:
   (ia) a crosslinked hyaluronic acid; or (ib) a cohesive crosslinked HA carrier produced by crosslinking hyaluronic acid with by divinylsulfone in alkaline environment, and then precipitated the crosslinked hyaluronic acid with ethanol; and
   (iia) PLA microspheres; (iib) PEG-PLA microspheres; (iic) PLA-PCL microspheres; or (iid) crosslinked HA microspheres produced by emulsified crosslinking reaction using ε-polylysine and 4-methylmorpholine hydrochloride (DMTMM) as the crosslinker in the presence of organic oil (such as olive oil or silicone oil) under stirring;
   for example, the composition comprises (ia) and (iia); (ia) and (iib); (ia) and (iic); or (ib) and (iid).
11 A method for the preparation of the composition of any one of items 1-10 comprising:
   combining the HA carrier with the slowly resorbable particles, spheres and granules.
12. The method of item 11, wherein the slowly resorbable particles, spheres and granules are combined with the HA carrier by utilizing vacuum planetary mixer to form an injectable homogeneous gel, such as at a revolution speed of 200 rpm˜1400 rpm and at an autorotation speed of 100 rpm˜700 rpm; and/or in a mixing time of 10˜30 minutes with vacuum; and/or
   wherein the slowly resorbable particles, spheres and granules are added to ethanol or methanol or acetone precipitation of the HA carrier and re-solubilized by hyaluronic acid solution or 0.9% sodium chloride solution or PBS buffer to form a homogeneous injectable gel.
13. The method of item 11, further comprising:
   adding a chemical agent to the mixture of the slowly resorbable particles, spheres and granules and the HA carrier to make crosslink or modification,
   such as by adding a chemical agent selected from the group consisting of divinylsulfone, glutaraldehyde, 1,4-butanediol diglycidyl ether or p-phenylene biscarbodiimide or 1,2,7,8-diepoxyoctane or oligomers rich in amino groups, poly-lysine or poly-arginine or γ-polyglutamic acid; and/or
   dialysis against 0.9% sodium chloride solution or PBS buffer, to obtain a homogeneous injectable composition.
14. A method for augmenting soft tissue in a subject in need thereof, comprising injecting the composition of any of items 1-10 to the site in need of the augment.
15. The method of item 14, wherein the composition is injected into soft tissue to correct soft tissue deficiencies; and/or
   the composition is injected into dermis to correct soft tissue deficiencies including wrinkles, dermal folds, dermal laxity, unevenness, facial emaciation, fat atrophy, cheek depression, eye socket depression, or a combination thereof.
16. The method of item 14 wherein the composition is injectable through a 25˜27 gauge needle or cannula, such as a 25, 27 or 30 gauge needle or cannula.
17. The composition of item 1, wherein the crosslinked hyaluronic acid is prepared by a method disclosed in CN111234271B.
18. The composition of item 1, wherein the crosslinked HA microspheres are obtained through emulsified crosslinking reaction, for example prepared by a method disclosed in CN109224127B.

The examples set forth below are intended to illustrate the invention without limiting its scope.

Publications cited herein and the materials for which they are cited are hereby specifically incorporated by reference in their entireties. All reagents, unless otherwise indicated, were obtained commercially. All parts and percentages are by weight unless stated otherwise. An average of results is presented unless otherwise stated. The abbreviations used herein are conventional, unless otherwise defined.

### EXAMPLES

### Example 1. Preparation of Crosslinked Hyaluronic Acid/PLA Composition

For initial evaluation, commercial samples of crosslinked hyaluronic acid were obtained. The clear, viscous, crosslinked hyaluronic acid was supplemented with poly-L-lactic acid crystals procured from PolySciences. Crystals were milled and screened to provide a size distribution from 25-45 μm in diameter. Homogenous mixing was conducted in a cleanroom environment by slowly passing the mixture between two syringes to avoid formation of air bubbles. Centrifugation was conducted at 3500 rpm to assure removal of air bubbles. The HA-PLA syringes were placed at 2-8° C. for storage.

### Example 2. Cohesive Crosslinked HN PEG-PLA and PLA-PCL Composites

For further improved formulation, cohesive crosslinked hyaluronic acid gel was obtained according to CN111234271B (cf. Example 2). 2˜3% (w/v) hyaluronic acid (MW>1300 kDa) solution was crosslinked by divinylsulfone (DVS) in alkaline environment (2% NaOH solution) under 40° C. for 2.5 hours. After the reaction, 95% ethanol solution was added slowly until white precipitate was precipitated. The white precipitate was washed 5-10 times with the same concentration of ethanol solution and filtered to obtain white powder. The cohesive crosslinked sodium hyaluronate gel was obtained by dissolving the white powder in PBS (pH=7.0˜7.4) and sterilized by moist heat sterilization.

PEG-PLA microsphere (25-50 μm diameter) was obtained from Evonik RESOMER®LP t 16 and sterilized by EtO. PEG-PLA microsphere and sterilized cohesive HA gel (v/v ratio: 25˜45%) were mixed by THINKY Mixer in a container with a cooler adapter at 1000 rpm revolution speed (autorotation speed is half of revolution speed) for 10 minutes under aseptic process. A homogeneous cohesive HA/PEG-PLA composite without any bubble was obtained.

Preparation of homogeneous cohesive HA/PLA-PCL composite was the same except that PEG-PLA microspheres were replaced by PLA-PCL microspheres (Evonik RESOMER® LC 703 S).

### Example 3. Cohesive Crosslinked HA with Crosslinked HA Microsphere Composite

For further improved formulation, cohesive crosslinked hyaluronic acid gel was obtained according to CN111234271B (cf. Example 2). 2˜3% (w/v) hyaluronic acid (MW>1300 kDa) solution was crosslinked by divinylsulfone (DVS) in alkaline environment (2% NaOH solution, pH=8.5˜10) under 40° C. for 2.5 hours. After the reaction, 95% ethanol solution was added slowly until white precipitate was precipitated. The white precipitate was washed 5-10 times with the same concentration of ethanol solution and filtered to obtain white powder. The cohesive crosslinked sodium hyaluronate gel was obtained by dissolving the white powder in PBS (pH=7.0˜7.4) and sterilized by moist heat sterilization.

Crosslinked HA microspheres were obtained through emulsified crosslinking reaction (prepared according to Examples 1-2 of CN109224127B). In particular, 1˜3% hyaluronic acid (MW>1500 kDa) solution (in PBS, pH7.2) and crosslinker ε-polylysine and 4-methylmorpholine hydrochloride (DMTMM) in 0.9% sodium chloride solution at the same mole ratio with HA were mixed and reacted in 5 times volume of organic oil silicone oil and Span 80 mixture (v:v=1.0˜2.0:25) and stirred at 1000 rpm to 1500 rpm under 30˜45° C. for over 24 hours. Stirring in higher speed produced microspheres with smaller diameter. The additional organic oils are washed by n-hexane and ethyl acetate for 3 times and absolute ethanol for 3 times. The crosslinked HA microspheres were gathered through centrifugation at 8,000˜10,000 rpm for 10 min. After rehydration by PBS, crosslinked HA microsphere suspension was filtered to gather the right size (20˜50 μm) of microspheres. White powder of HA microspheres was obtained after ethanol precipitation.

Cohesive crosslinked HA gel dehydrated powder and crosslinked HA microsphere dehydrated powder were mixed at (V/V=1:1) and resolubilized in PBS to obtained cohesive crosslinked HA with crosslinked HA microsphere composite. The composite was sterilized by moist heat and store as a suspension form at 2˜35° C. for use.

### Example 4. In Vitro Degradation of Cohesive Crosslinked HA with Crosslinked HA Microsphere Composite

0.5 g (about 0.5 mL in filler) of cohesive crosslinked HA with crosslinked HA microsphere composite prepared in Example 3 (V/V=1:1) was added to 7.5 mL 300 U/mL hyaluronidase (Sigma) in PBS (pH=7.0˜7.2) and reacted under 37° C. for 8 hours. After centrifugation at 10,000 rpm for 15 min, no gel or microspheres was found (visually or filtrated by microfilters) in reaction solution and the composite was totally degraded by hyaluronidase.

In clinical practice, the enzyme hyaluronidase sterile solution is used to degrade hyaluronic acid and can therefore be injected into soft tissue to reduce suboptimally placed HA fillers or to reverse local ischemic complications. Slowly absorbable polymer microspheres are able to provide long-term effect, however, it is very hard to remove after implantation. Crosslinked HA microsphere may have similar longevity, and its reversibility with hyaluronidase will improve the safety of the formulation.

### Example 5. Evaluation of Crosslinked HA/Microsphere Composites in Animals

A one-month animal study was performed to examine tissue reactivity to the hyaluronic acid/PLA formulation prepared in Example 1 and a commercial hyaluronic acid control (Juvederm). Tests were conducted in one healthy New Zealand White Rabbit and in healthy mice. The rabbit model received injections of HA/PLA in the ear. Mice received injections in the back dermis. After 30 days, animals were euthanatized and implant sites were removed for fixation, sectioning, and histopathological evaluation at Mass Histology Service in Worcester, Mass. Sections were stained with H&E, Trichrome Blue, and Von Geison's (elastic fiber network). All stained specimens were evaluated, and images were prepared and a pathology report was drafted.

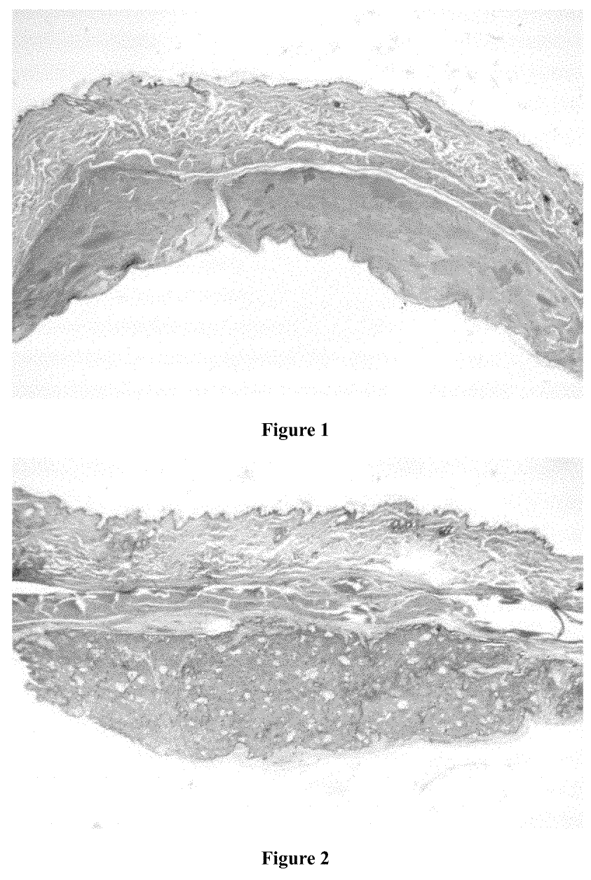

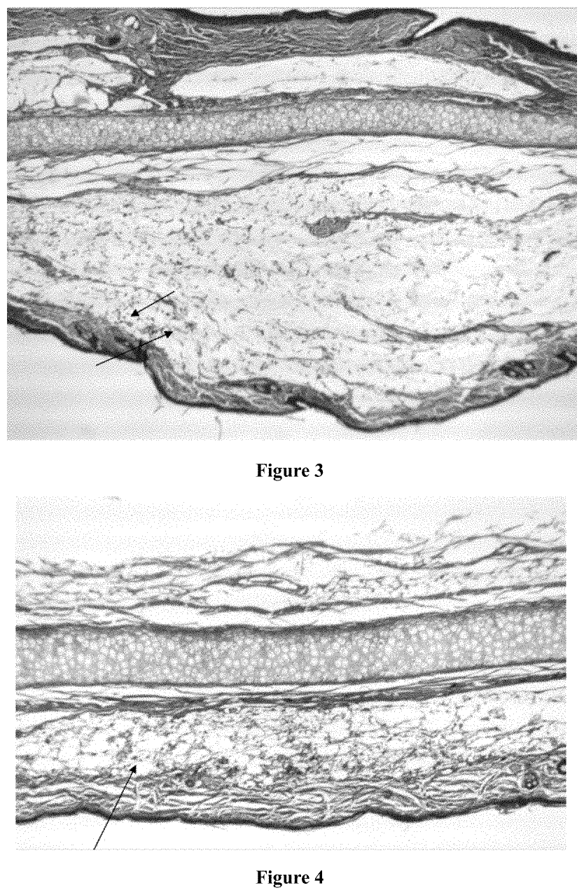

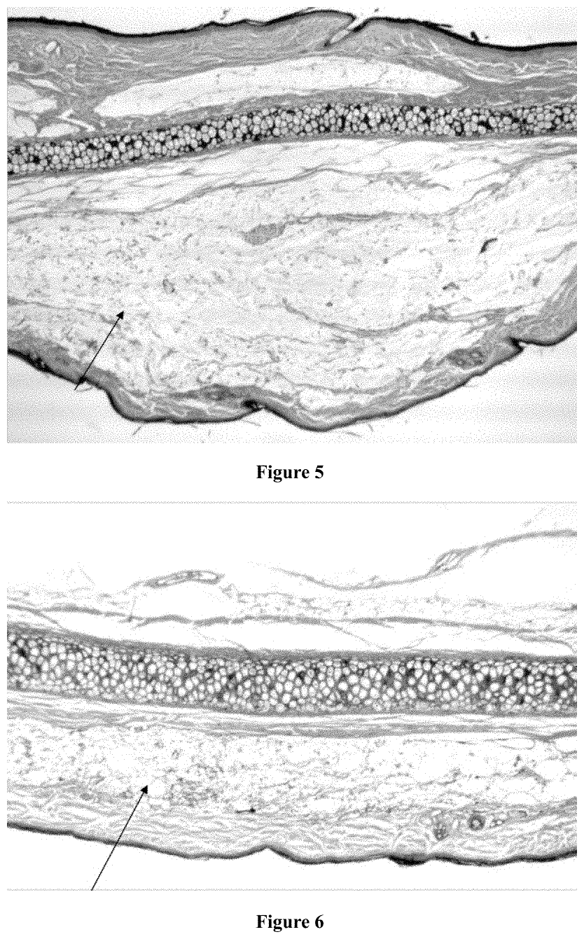

Histological evaluation of HA and HA+PLA implants after one month were clearly visible with limited evidence of inflammation (FIGS. 1 and 2); evidence of new collagen fiber formation throughout the implants and in the surrounding fibrous capsule in the rabbit explants were observed (FIGS. 3 and 4). Van Geison's staining did not show evidence of elastic tissue associated with the implants (FIGS. 5 and 6). Histological evaluation of HA+PLA in the rabbit model showed evidence of scattered collagen fibers throughout the implant (expected since the injection was composed of intact PLA in the HA carrier).

With the degradation of HA, macrophages and myofibroblasts integrated into the space and the PLA microspheres were enveloped by surrounding tissues and macrophages and myofibroblasts integrated into the space where HA degraded. Neocollagen and neotissue formation are observed with the stimulation of PLA microspheres.

As compared to the effect of the control HA (which is a successful commercialized product), the effect of HA+PLA implants of the present invention is at least as good as or even better than that of the control.

### CONCLUSIONS

All implants showed low to very low levels of inflammatory reactivity. HA and HA+PLA implants in the rabbit model showed small early collagen fibers scattered throughout the implant indicating some host tissue integration or stimulation of neocollagensis (see FIG. 1: HA control & FIG. 2: HA/PLA implant).

PLLA, calcium hydroxylapatite (CaHA), poly(caprolactone) (PCL) microspheres with carriers such as Carboxyl Methyl Cellulose (CMC) and normal saline are widely used as collagen stimulators in soft tissue augmentation. Most of these carrier materials undergo fast diffusion or dispersing after implantation. Acute inflammation to the microsphere causes foreign body giant cells gathering in the spaces between and on the surfaces of the microspheres, and complications such as swelling, firmness, lumps/bumps, pain, redness, itching will occur.

As compared to cohesive crosslinked HA gel having limited swelling and dispersion in vitro and in vivo, cohesive crosslinked HA with slowly degradable polymer of the present disclosure reduces specific surface area of the microspheres when implanted, avoids microspheres displacement and diffusion in tissues and further decreases the level of inflammatory reactivity.

As compared to cohesive crosslinked HA usually having longevity because of the lower specific surface area, cohesive crosslinked HA with slowly degradable polymer of the present disclosure is much safer and longer lasting. After injection of the composite, crosslinked HA is absorbed by the body first, and neo-collagen under the stimulus of polymer microspheres replaces the implant, augmenting skin deficiency, and finally after the slow degradation of the polymer molecules, can still maintain implantation effect. Although the slowly absorbable microspheres are considered biocompatible, it cannot be degraded fast in vivo. Crosslinked HA gel with crosslinked microsphere composite can be easily degraded by hyaluronidase and make the implantation of the composite reversible like normal crosslinked HA gel.

Without intending to be bound by theory, the ordered arrangement of particles provides a 3D spatial structure for immune-related cells in soft tissue, inducing neocollagenesis. Hyaluronan with high molecular weight greater than 1,000 kDa exert antiangiogenic, immunosuppressive, and anti-inflammatory effects. Crosslinked or modified hyaluronic acid usually has high molecular weight and is immunologically inert and thus reducing the irritation of implantation to a lower level. Combination of crosslinked HA with slowly absorbable microspheres can regulate immunity from mechanical and spatial tendency, shortening acute inflammatory period after implantation, and improve the safety, longevity and effect of the composition in soft tissue augmentation.

### OTHER EMBODIMENTS

Although the present invention has been described with reference to preferred embodiments, one skilled in the art can easily ascertain its essential characteristics and without departing from the spirit and scope thereof, can make various changes and modifications of the invention to adapt it to various usages and conditions. Those skilled in the art will recognize or be able to ascertain using no more than routine experimentation, many equivalents to the specific embodiments of the invention herein. Such equivalents are intended to be encompassed in the scope of the present invention.

All references, including patents, publications, and patent applications, mentioned in this specification are herein incorporated by reference in the same extent as if each independent publication, patent or patent application was specifically and individually indicated to be incorporated by reference.

### REFERENCES

### Publications

M. Rehakova et. al. J Biomed Mater Res, 1996 30:369
Y. K. Lin et. al. J Biomater Appl, 2007 21:265

### Patents

U.S. Pat. No. 5,824,333;
U.S. Pat. No. 7,314,636
U.S. Pat. No. 7,887,599
U.S. Pat. No. 8,455,459
U.S. Pat. No. 8,580,289
U.S. Pat. No. 9,480,775
CN 105879124
CN 109503864B
CN 111234271B
CN 109224127B
US 2008/0188416
US 2009/0117188
US 2010/0217403
US 2010/0210588
US 2011/0087152

We claim:

1. A composition for use in soft tissue augmentation comprising
(i) an HA carrier, wherein the carrier is selected from hyaluronic acid, modified hyaluronic acid, or crosslinked hyaluronic acid, or salts thereof (such as sodium salt); and
(ii) slowly resorbable particles, spheres and granules.

2. The compositions of claim 1, wherein the HA carrier has an molecular weight of from 1,000 to 10,000,000 Dalton, and/or
the HA carrier is derived from animal tissue or microorganism fermentation.

3. The composition of claim 1, wherein the modified hyaluronic acid or crosslinked hyaluronic acid are modified or crosslinked by divinylsulfone, glutaraldehyde, 1,4-butanediol diglycidyl ether, p-phenylene biscarbodiimide or 1,2,7,8-diepoxyoctane, or oligomers rich in amino groups (such as poly-lysine or poly-arginine or γ-polyglutamic acid); and/or
the concentration of modified hyaluronic acid or crosslinked hyaluronic acid is from 12 mg/mL to 30 mg/mL, preferably from 16 mg/mL to 24 mg/mL; and/or
the modified hyaluronic acid or crosslinked hyaluronic acid is cohesive.

4. The composition of claim 1, wherein the crosslinked hyaluronic acid microspheres are coated with bio-degradable polymers, such as poly-L-lactide (PLA), polyethylene glycol (PEG), or PLGA, or poly(p-dioxanone) (PDO); and/or
wherein the crosslinked hyaluronic acid microspheres are produced by emulsified crosslinking reaction, double emulsion evaporation method, microfluidic crosslinking reaction, or stamp formation; and/or
crosslinker of the crosslinked hyaluronic acid microspheres is selected from the group consisting of divinylsulfone, glutaraldehyde, 1,4-butanediol diglycidyl ether or p-phenylene biscarbodiimide or 1,2,7,8-diepoxyoctane or oligomers rich in amino groups (such as poly-lysine or poly-arginine or γ-polyglutamic acid; and/or
wherein the crosslinked hyaluronic acid microsphere has a particle size of particle size: 5˜150 μm, preferrably 20˜50 μm.

5. The composition of claim 1, wherein the slowly resorbable particles, spheres and granule spheres are selected from particles, spheres or granules of poly-L-lactide (PLA), PEG-PLA copolymer or poly-L-lactide-hydroxyapatite, polyglycolic acid (PGA), poly-L-lactide-hydroxyapatite, crosslinked hyaluronic acid microspheres.

6. The composition of claim 1, wherein the slowly resorbable particles, spheres or granules are particles, spheres or granules of polylactide and polyglycolide polymers and copolymers (PLGA); and/or particles, spheres or granules of poly ε-caprolactone (PCL) or PCL-PLA copolymer or poly ε-caprolactone-hydroxyapatite microspheres; and/or poly(p-dioxanone) (PDO) or poly(p-dioxanone)-hydroxyapatite.

7. The composition of claim 1, wherein the slowly resorbable particles, spheres or granules are produced through spray-precipitation technique, emulsion, double emulsion evaporation method, microfluidic reaction, Solid-Gel process, melt extrusion technique or stamp formation; and/or are sterilized through heat moist sterilization, gamma irradiation or ethylene oxide sterilization; and/or
wherein the slowly resorbable particles, spheres or granules have a diameter of from 5 to 150 μm, preferably 20 to 50 μm.

8. The composition of claim 1, further comprising additives, for example those selected from the group consisting of:
local anesthesia drugs such as lidocaine, procaine, etc, preferably in a concentration of from 0.1% to 0.5% by weight; and/or
polyols stabilizers, such as glycerin, mannitol, butanediol, sorbitol, preferably in a concentration of from 0.1 to 5% by weight; and/or
a stabilizer with chelating ability, such as EDTA, EGTA, citric acid, sodium citrate, preferably in a concentration of from 0.1 to 5% by weight; and/or
a sulfur stabilizer or dissolution promotor, such as Chondroitin Sulfate Sodium (CS), Gluscosamine Sulphate (GS) or Methyl sulfonyl methane (MSM), preferably in a concentration of from 0.1% to 5% by weight; and/or
wherein soluble small molecules are added through dialysis process.

9. The composition of claim 1, wherein the amount of HA carrier in the composition is from 0.1% to 55% by volume; and/or
the amount of the slowly resorbable particles, spheres or granules in the composition is from 0.1% to 50% by volume, preferably from 1% to 20% by volume; and/or
the additive in the composition is from 0.1% to 10% by weight, preferably from 0.1% to 5% by weight.

10. The composition of claim 1, wherein the composition comprises:
(ia) a crosslinked hyaluronic acid; or (ib) a cohesive crosslinked HA carrier produced by crosslinking hyaluronic acid with by divinylsulfone in alkaline environment, and then precipitated the crosslinked hyaluronic acid with ethanol; and
(iia) PLA microspheres; (iib) PEG-PLA microspheres; (iic) PLA-PCL microspheres; or (iid) crosslinked HA microspheres produced by emulsified crosslinking reaction using ε-polylysine and 4-methylmorpholine hydrochloride (DMTMM) as the crosslinker in the presence of organic oil (such as olive oil or silicone oil) under stirring;
for example, the composition comprises (ia) and (iia); (ia) and (iib); (ia) and (iic); or (ib) and (iid).

11. A method for the preparation of the composition of claim 1 comprising:
combining the HA carrier with the slowly resorbable particles, spheres and granules.

12. The method of claim 11, wherein the slowly resorbable particles, spheres and granules are combined with the HA carrier by utilizing vacuum planetary mixer to form an injectable homogeneous gel, such as at a revolution speed of 200 rpm˜1400 rpm and at an autorotation speed of 100 rpm˜700 rpm; and/or in a mixing time of 10˜30 minutes with vacuum; and/or
wherein the slowly resorbable particles, spheres and granules are added to ethanol or methanol or acetone precipitation of the HA carrier and re-solubilized by hyaluronic acid solution or 0.9% sodium chloride solution or PBS buffer to form a homogeneous injectable gel.

13. The method of claim 11, further comprising:
adding a chemical agent to the mixture of the slowly resorbable particles, spheres and granules and the HA carrier to make crosslink or modification,
such as by adding a chemical agent selected from the group consisting of divinylsulfone, glutaraldehyde, 1,4-butanediol diglycidyl ether or p-phenylene biscarbodiimide or 1,2,7,8-diepoxyoctane or oligomers rich in amino groups, poly-lysine or poly-arginine or γ-polyglutamic acid; and/or
dialysis against 0.9% sodium chloride solution or PBS buffer, to obtain a homogeneous injectable composition.

14. A method for augmenting soft tissue in a subject in need thereof, comprising injecting the composition of claim 1 to the site in need of the augment.

15. The method of claim 14, wherein the composition is injected into soft tissue to correct soft tissue deficiencies; and/or
the composition is injected into dermis to correct soft tissue deficiencies including wrinkles, dermal folds, dermal laxity, unevenness, facial emaciation, fat atrophy, cheek depression, eye socket depression, or a combination thereof.

16. The method of claim 14 wherein the composition is injectable through a 25˜27 gauge needle or cannula, such as a 25, 27 or 30 gauge needle or cannula.
